(19) United States
(12) Patent Application Publication
(10) Pub. No.: US 2010/0179650 A1
(43) Pub. Date: Jul. 15, 2010

(54) SEPARATE AORTIC ARTIFICIAL VALVE LEAFLET AND TEMPLATE FOR PREPARATION THEREOF

(76) Inventor: Meong-Gun Song, Seoul (KR)

Correspondence Address: MORRISON & FOERSTER LLP, 755 PAGE MILL RD, PALO ALTO, CA, 94304-1018, US

(21) Appl. No.: 12/521,323

(22) PCT Filed: Feb. 11, 2008
(86) PCT No.: PCT/KR08/00781

§ 371 Date: Mar. 8, 2010

(30) Foreign Application Priority Data
Feb. 6, 2007 (KR) 10-2007-0012069

(51) Int. Cl. A61F 2/24

(52) U.S. Cl. 623/2.12

(57) ABSTRACT

Provided is an artificial aortic valve leaflet useful in the surgical treatment of aortic valve disorders through the reconstruction of aortic leaflets. The artificial aortic valve leaflet comprising a plane having four sides, three sides of which are linear and a remaining side of which is curved. The distance between a center of the curved side and a center of a side opposite the curved side ranges from 0.35 to 0.70 times a length of the side opposite the curved side. The length of each side adjacent to the curved side ranges from 0.20 to 0.50 times the length of the side opposite the curved side.

### TECHNICAL FIELD

The present invention relates to an artificial aortic valve leaflet useful in the surgical treatment of aortic valve disorders through the reconstruction of aortic leaflets. Also, the present invention is concerned with a template for use in the preparation of the artificial aortic valve leaflet for the reconstruction of deformed aortic leaflets. More particularly, the present invention relates to a template composed of an upper plate and a lower plate and, optionally, grips.

### BACKGROUND ART

There are four valves in the heart that maintain the unidirectional flow of blood. Of them, two, known as the mitral valve and the aortic valve, are on the left side of the heart, through which oxygenated blood flows, and the other two, called the tricuspid valve and the pulmonic valve, are on the right side through which deoxygenated blood flows. Lying between the left ventricle and the aorta, the aortic valve is designed to stand the highest pressure in the heart. The aortic valve is normally comprised of three leaflets. In the most common congenital abnormality of the heart, the aortic valve has either two or four leaflets. Each leaflet is anchored through the aortic annulus to the wall of the aorta. There is a space called the “sinus of valsalva” between the leaflet and the wall of the aorta.

For the normal aortic valve, there are generally three leaflets, named the left coronary aortic sinus, the right coronary aortic sinus, and the non-coronary aortic sinus. They get these names from the fact that the left and the right coronary artery originate from the right side of the sinus of valsalva, respectively, and that the non-coronary aortic sinus does not have a coronary artery ostium of from the sinus of valsalva.

In anatomy, a commissure refers to a place where two leaflets come together. Thus, the normal aortic valve also has three commissural sites near the wall of the aorta. Each leaflet is not flat, but is concave, like a rose petal, when viewed from the sinus of valsalva. The aortic valve, the aortic annulus, the aortic wall, and the sinus of valsalva are collectively called the aortic root. The aortic root has a shape of truncated cone in which the aortic annulus and the sinotubular junction each correspond to the lower margin and the upper margin of that, with the aortic valve located therebetween. The aortic valve opens and closes by the twisting and tilting of the aortic muscles. The contraction of the left ventricle opens the aortic valve, allowing blood to move toward the aorta. When ventricular systole ends, the pressure in the left ventricle rapidly drops, and thus the aortic pressure forces the leaflets to engage with one another, resulting in the closure of the aortic valve.

For complete closure of the aortic valve, the sinotubular junction must maintain its diameter, under which the leaflets are in perfect engagement with one another. Aortic valve disorders therefore largely come from lesions either in the leaflets themselves or in the aortic wall. Between them, there are associated lesions as well, like a spectrum. In the early stage of an aortic valve disorder, a lesion may only be confined in a leaflet. Even in this case, the lesion, for the most part, incurs structural modification in the adjacent aortic root region with the lapse of time. On the other hand, the imperfect closure of the leaflets may be caused by lesions in the aortic root region, such as dilatation, dissection.

Isolated aortic valve disorders refer to aortic valve disorders with lesions only confined in leaflets themselves, and can be classified into aortic stenosis and aortic regurgitation. Some patients suffer from a combined aortic valve disorder with the concurrent occurrence of the above two lesions. In contrast, when a lesion occurs in the aortic root, all of the aortic valve leaflets undergo functional insufficiency with the additional concomitant effects of dilatation, dissection and inflammation in the aortic root and the neighbor ascending aorta. In this case, it is reasonable to regard the disorder as a complex disorder associated with aortic disorders, rather than as a single aortic valve disorder.

Accordingly, a preferred requisite for the treatment of aortic valve disorders is the examination of whether the aortic valve disorders are attributed to the valve alone or are associated with the disorders of the neighboring ascending aorta and the aortic root. As for the single disorders of the aortic valve alone, replacement with artificial valves has been generally used as a standard therapy, thus far. There are two kinds of artificial heart valves: mechanical valves and tissue valves.

After aortic valve replacement with mechanical valves, the patients should take an anticoagulant medication. On the other hand, tissue valves have the problem of requiring repeat surgery due to the low durability thereof. Hemodynamically, both types of artificial heart valves are more disadvantageous than autografts in that the actual cross section area is reduced.

Despite these problems, artificial valve replacement has generally been employed because it is difficult to apply valve repair to the treatment of aortic valve disorders. Further, in most cases of complex aortic valve disorders associated with aortic disorders, not only are the aortic valves replaced with artificial valves, but also the neighboring aortic vessels are replaced with artificial vessels. For the convenience of cardiothoracic surgery, in practice, a composite graft, in which an artificial valve is combined with an artificial vessel, is widely employed. Recently, a new technique (David technique), in which the aorta is replaced with an artificial vessel while an autograft valve is saved without replacement, has been applied in some cases. The technique, however, is problematic in that the ventricular function is difficult to sustain for a long period of time because the function of the aortic root is not recovered. In other conditions, many repeated attempts have been made to reconstruct the aortic valve. For example, Duran et al. suggested aortic leaflet extension using a strip of autologous pericardium. However, because this is designed for all three aortic valve leaflets, all of them must be removed even if lesions are present only on one or two of them. In addition, the application of this technique is limited only to isolated aortic valve disorders.

Moreover, short- and long-term operation results were found to be very poor because the diameter of the sinotubular junction is not fixed

In order to overcome the problems encountered with the artificial aortic valve replacement and the artificial vessel replacement for the aortic root, Dr. Song, from South Korea, has recently developed a novel surprising surgical technique, called Comprehensive Aortic Root & Valve Repair (CARVAR), by which the aortic root as well as the aortic valve are reconstructed from autologous tissue with good surgical results. CARVAR can be divided largely into three levels: Level 1, in which the aortic annulus, a lowermost part of the aortic root, is prevented from; enlargement Level 2, in which the sinotubular junction, an uppermost part of the aortic root, is fixed so as to prevent the dilatation thereof; and Level 3, in which the aortic valve leaflets are repaired and engaged with each other.

Without taking Levels 1 and 2 into consideration, conventional aortic valvuloplasty techniques have been conducted with low success rates. In contrast, the CARVAR technique, developed by Dr. Song, in which the Levels 1 and 2 are determined with a given size, followed by repairing the engagement of the leaflets based on the determination, can result in great success in valvuloplasty. Given that the aortic root region is figured as a truncated cone shape, when the length of the upper and the lower end thereof are determined, the lengths of the leaflets located there between fall within the range between the lengths of the upper and the lower end. This can be applied to the determination of the size of individual leaflets. Thus, even if the number of leaflets is not three but two, their sizes can be determined within the mathematical range.

### DISCLOSURE OF INVENTION

Accordingly, the present invention has been made keeping in mind the above problems occurring in the prior art. An object of the present invention is to provide a template for use in precisely and rapidly preparing artificial aortic valve leaflets tailored for the aortic valves of patients by using a biomaterial compatible with the aortic valve leaflets, and the artificial aortic valve leaflets prepared.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a plan view illustrating an upper plate and a lower plate of a template according to the present invention;

FIG. 2 is a plan view illustrating a grip-coupled template according to the present invention; and

FIG. 3 is a view illustrating the use of a template according to the present invention.

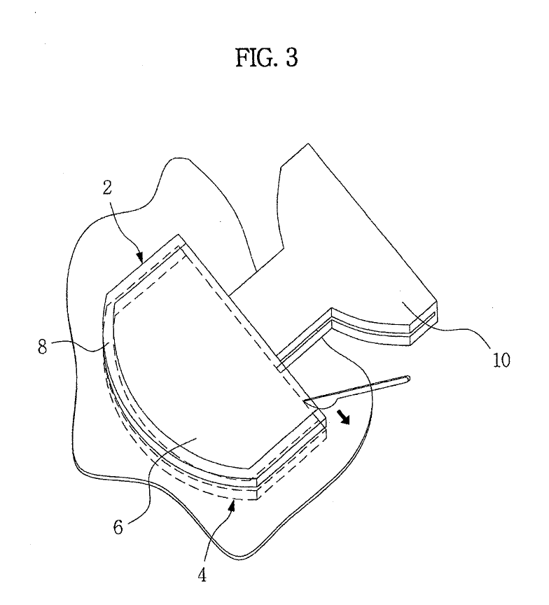

### BEST MODE FOR CARRYING OUT THE INVENTION

As a rule, surgical technique for reconstructing dysfunctioning aortic valve leaflets by using a biomaterial is selected from the following three methods:

(1) “complete-leaflet replacement,” applied to the case where lesions are spread over the entire leaflet, in which the entire leaflet is completely excised and a new artificial leaflet is attached to the annulus;

(2) “partial leaflet resection and extension,” applied to the case where a leaflet has a thickened lesion at an edge or is partially ruptured, in which part, but not all, of the leaflet is excised and a new artificial leaflet is attached; and

(3) “simple leaflet extension,” applied to the case where a leaflet shows prolapse at a central cuspal reclining, even though it moves smoothly and is not thickened, in which an artificial leaflet is attached, without the resection of the leaflet, to increase the area of the central commissural site.

Among these cases, the method (1), called “complete-leaflet replacement,” is the most difficult one to perform and takes a lot of time. In addition, it has a relatively high risk of rupture because high tension is applied to the sutured sites. Therefore, it is convenient to use the method (2) or (3) whenever possible.

The present invention is directed to a template used to prepare each artificial aortic valve leaflet. Here, the dimensions of the template may be different from those of the artificial aortic valve leaflet depending on the surgical methods (1), (2) and (3). The artificial aortic valve leaflet used to treat aortic valve disorders will be described first. The individual artificial aortic valve leaflet template comprises a plane having four sides. Among the four sides, three sides are linear, while one side is curved. The distance between the center of the curved side and the center of the side opposite the curved side is within a range from 0.35 to 0.70 times the length of the side opposite the curved side. The length of each side adjacent to the curved side ranges from 0.20 to 0.50 times the length of the side opposite the curved side. An extension part for a suturing space is formed along outer edges of the curved side and the sides adjacent to the curved side.

More preferably, the present invention relates to a individual artificial aortic valve leaflet used to treat aortic valve disorders, which comprises a plane having four sides, three sides of which are linear, and one side of which is curved. Here, the distance between the center of the curved side and the center of the side opposite the curved side ranges from 0.65 to 0.70 times the length of the side opposite the curved side. The length of each side adjacent to the curved side ranges from 0.40 to 0.50 times the length of the side opposite the curved side. An extension part for a suturing space is formed along outer edges of the curved side and the sides adjacent to the curved side.

Further, the present invention relates to an individual artificial aortic valve leaflet used to treat aortic valve disorders, which comprises a plane having four sides, three sides of which are linear, and one side of which is curved. Here, the distance between the center of the curved side and the center of the side opposite the curved side is calculated by subtracting a value ranging from 1 mm to 5 mm from a value ranging from 0.65 to 0.70 times the length of the side opposite the curved side. The length of each side adjacent to the curved side is calculated by subtracting a value ranging from 1 mm to 5 mm from a value ranging from 0.40 to 0.50 times the length of the side opposite the curved side. An extension part for a suturing space is formed along outer edges of the curved side and the sides adjacent to the curved side.

The present invention also relates to an individual artificial aortic valve leaflet used to treat aortic valve disorders, which comprises a plane having four sides, three sides of which are linear and one side of which is curved. Here, the length between the center of the curved side and the center of the side opposite the curved side ranges from 0.35 to 0.40 times the length of the side opposite the curved side. The length of each side adjacent to the curved side ranges from 0.20 to 0.25 times the length of the side opposite the curved side. An extension part for a suturing space is formed along outer edges of the curved side and the sides adjacent to the curved side.

At this time, the extension part of the artificial aortic valve leaflet according to the present invention preferably has a width ranging from 1 mm to 2 mm.

Further, the present invention relates to a template for use in the preparation of the individual artificial aortic valve leaflet comprises a plane having four sides. Among the four sides, three sides are linear, while one side is curved. The distance between the center of the curved side and the center of the side opposite the curved side is within a range from 0.35 to 0.70 times the length of the side opposite the curved side. The length of each side adjacent to the curved side ranges from 0.20 to 0.50 times the length of the side opposite the curved side. The upper plate has an extension part formed along outer edges of the curved side and the sides adjacent to the curved side and the lower plate is the same as the upper plate.

More preferably, the present invention relates to a template for use in the preparation of the individual artificial aortic valve leaflet used to treat aortic valve disorders, which comprises a plane having four sides, three sides of which are linear, and one side of which is curved. Here, the distance between the center of the curved side and the center of the side opposite the curved side ranges from 0.65 to 0.70 times the length of the side opposite the curved side. The length of each side adjacent to the curved side ranges from 0.40 to 0.50 times the length of the side opposite the curved side. The upper plate has an extension part formed along outer edges of the curved side and the sides adjacent to the curved side and the lower plate is the same as the upper plate.

Further, the present invention relates to a template for use in the preparation of the individual artificial aortic valve leaflet used to treat aortic valve disorders, which comprises a plane having four sides, three sides of which are linear, and one side of which is curved. Here, the distance between the center of the curved side and the center of the side opposite the curved side is calculated by subtracting a value ranging from 1 mm to 5 mm from a value ranging from 0.65 to 0.70 times the length of the side opposite the curved side. The length of each side adjacent to the curved side is calculated by subtracting a value ranging from 1 mm to 5 mm from a value ranging from 0.40 to 0.50 times the length of the side opposite the curved side. The upper plate has an extension part formed along outer edges of the curved side and the sides adjacent to the curved side and the lower plate is the same as the upper plate.

The present invention also relates to a template for use in the preparation of the individual artificial aortic valve leaflet used to treat aortic valve disorders, which comprises a plane having four sides, three sides of which are linear and one side of which is curved. Here, the length between the center of the curved side and the center of the side opposite the curved side ranges from 0.35 to 0.40 times the length of the side opposite the curved side. The length of each side adjacent to the curved side ranges from 0.20 to 0.25 times the length of the side opposite the curved side. The upper plate has an extension part formed along outer edges of the curved side and the sides adjacent to the curved side and the lower plate is the same as the upper plate.

At this time, the extension part of the template according to the present invention preferably has a width ranging from 1 mm to 2 mm, and grips could be attached to the upper plate and the lower plate of the template in addition.

Further, the ratio of the lengths of the four sides of the plane is very important. Particularly, the ratio varies as follows, depending on whether the surgical technique for reconstructing dysfunctinging aortic valve leaflets is (1) complete-leaflet replacement, (2) partial leaflet resection and extension, or (3) simple leaflet extension.

In an embodiment of the present invention, the artificial aortic valve leaflet used to the complete-leaflet replacement adopts, as a reference length, the length of the side opposite the curved side of the plane. The length between the center of the curved side and the center of the side opposite the curved side preferably ranges from 0.65 to 0.70 times the reference length, and more preferably ⅔ times. The length of each side adjacent to the curved side preferably ranges from 0.40 to 0.50 times the length of the side opposite the curved side, and more preferably 4/9 times. At this time, the extension part for the suturing space is formed along the outer edges of the curved side and the sides adjacent to the curved side, and preferably has a width ranging from 1 mm to 2 mm.

In another embodiment of the present invention, the artificial aortic valve leaflet used to the partial leaflet resection and extension adopts, as a reference length, the length of the side opposite the curved side of the plane. The length between the center of the curved side and the center of the side opposite the curved side is preferably calculated by subtracting a value ranging from 1 mm to 5 mm from a value ranging from 0.65 to 0.70 times the reference length, and more preferably calculated by subtracting 3 mm from ⅔ times the reference length. The length of each side adjacent to the curved side is preferably calculated by subtracting a value ranging from 1 mm to 5 mm from a value ranging from 0.40 to 0.50 times the length of the side opposite the curved side, and more preferably calculated by subtracting 3 mm from 4/9 times the reference length. The extension part for the suturing space is formed along the outer edges of the curved side and the sides adjacent to the curved side, and preferably has a width ranging from 1 mm to 2 mm.

In another embodiment of the present invention, the artificial aortic valve leaflet used to the simple leaflet extension adopts, as a reference length, the length of the side opposite the curved side of the plane. The length between the center of the curved side and the center of the side opposite the curved side preferably ranges from 0.35 to 0.40 times the reference length, and more preferably 10/27 times the reference length. The length of each side adjacent to the curved side preferably ranges from 0.20 to 0.25 times the length of the side opposite the curved side, and more preferably 2/9 times the reference length. At this time, the extension part for the suturing space is formed along the outer edges of the curved side and the sides adjacent to the curved side, and preferably has a width ranging from 1 mm to 2 mm.

The artificial aortic valve leaflet according to the present invention can be prepared by cutting excess material extending beyond extension parts of upper and lower plates of the template using a pair of surgical scissors or a surgical knife after it is placed between the upper and lower plates of the template. The artificial aortic valve leaflet according to the present invention is made of a piece of biological material such as autologous pericardium, bovine or porcine pericardium, or the valve tissue, peritoneum, pleura or fascia of animal or man, which is fixed with glutaraldehyde in a flat shape.

In another embodiment of the present invention, template for use in the preparation of the artificial aortic valve leaflet used to the complete-leaflet replacement adopts, as a reference length, the length of the side opposite the curved side of the plane. The length between the center of the curved side and the center of the side opposite the curved side preferably ranges from 0.65 to 0.70 times the reference length, and more preferably ⅔ times the reference length. The length of each side adjacent to the curved side preferably ranges from 0.40 to 0.50 times the length of the side opposite the curved side, and more preferably 4/9 times the reference length. At this time, the extension part for the suturing space is formed along the outer edges of the curved side and the sides adjacent to the curved side, and preferably has a width ranging from 1 mm to 2 mm. The lower plate according to the present invention is formed the same as the upper plate.

In another embodiment of the present invention, a template for use in the preparation of the artificial aortic valve leaflet used to the partial leaflet resection and extension adopts, as a reference length, the length of the side opposite the curved side of the plane. The length between the center of the curved side and the center of the side opposite the curved side is preferably calculated by subtracting a value ranging from 1 mm to 5 mm from a value ranging from 0.65 to 0.70 times the reference length, and more preferably calculated by subtracting 3 mm from ⅔ times. The length of each side adjacent to the curved side is preferably calculated by subtracting a value ranging from 1 mm to 5 mm from a value ranging from 0.40 to 0.50 times the length of the side opposite the curved side, and more preferably calculated by subtracting 3 mm from 4/9 times the reference length. At this time, the extension part for the suturing space is formed along the outer edges of the curved side and the sides adjacent to the curved side, and preferably has a width ranging from 1 mm to 2 mm. The lower plate according to the present invention is formed the same as the upper plate.

In another embodiment of the present invention, a template for use in the preparation of the artificial aortic valve leaflet used to the simple leaflet extension adopts, as a reference length, the length of the side opposite the curved side of the plane. The length between the center of the curved side and the center of the side opposite the curved side preferably ranges from 0.35 to 0.40 times the reference length, and more preferably 10/27 times the reference length. The length of each side adjacent to the curved side preferably ranges from 0.20 to 0.25 times the length of the side opposite the curved side, and more preferably 2/9 times the reference length. At this time, the extension part for the suturing space is formed along the outer edges of the curved side and the sides adjacent to the curved side, and preferably has a width ranging from 1 mm to 2 mm. The lower plate according to the present invention is formed the same as the upper plate.

In the template according to the present invention, the lower plate 4 has the same shape as the upper plate 2. Any material can serve as the material for the upper and lower plates 2 and 4, as long as it is harmless to the human body. Preferably, polycarbonate is used. Further, a grip may be additionally coupled to the upper and lower plates of the template according to the present invention.

Hereinafter, the present invention will be described in detail with reference to the attached drawings. The following description is merely exemplary in nature, and is in no way intended to limit the present invention.

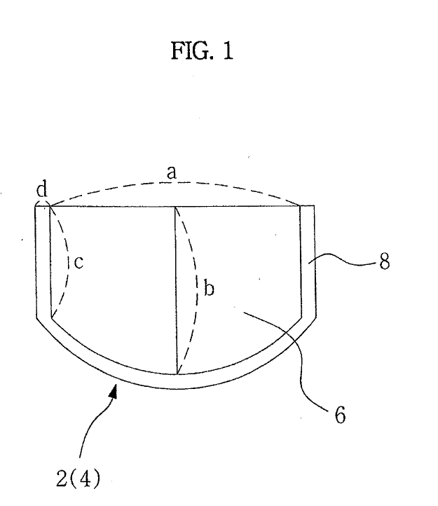

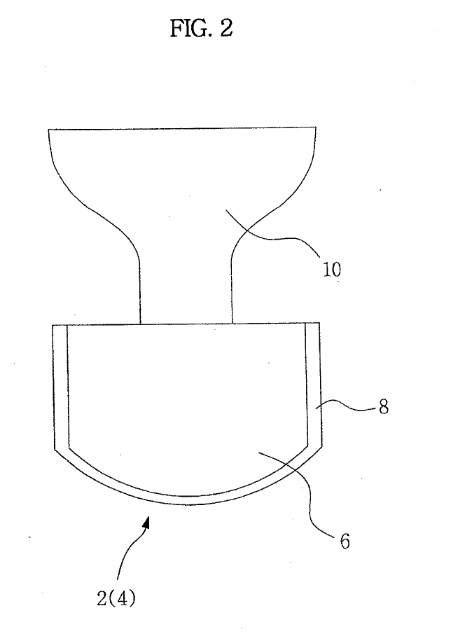

FIG. 1 is a plan view illustrating the upper and lower plates of a template according to the present invention, and FIG. 2 is a plan view illustrating a template, to which a grip is coupled, according to the present invention.

As illustrated in FIG. 1, the template for preparing an artificial aortic valve leaflet according to the present invention comprises an upper plate 2 and a lower plate 4, from a macroscopic point of view.

Now, the present invention will be described in greater detail with reference to the surgical technique of the aortic valve leaflet.

(1) In the complete-leaflet replacement of the first embodiment of the present invention, the upper plate 2 of the template according to the present invention comprises a flat plate 6 having four sides. The length of each side of the flat plate 6 is defined by adopting, as a reference length, the length a of the side opposite the curved side of the flat plate 6. The length b, which is the distance between the center of the curved side and the center of the side opposite the curved side, is from 0.65 to 0.70 times (about ⅔ times) the reference length a. The length c of each side adjacent to the curved side is from 0.40 to 0.50 times (about 4/9 times) the length a of the side opposite the curved side.

Here, in order to provide a margin for suturing the artificial aortic valve leaflet when the artificial aortic valve leaflet is prepared, an extension part 8 is formed along the outer edges of the curved side of the flat plate 6 and the sides adjacent to the curved side. The extension part 8 is formed along the outer edges of the curved side of the flat plate 6 and the sides adjacent to the curved side so as to extend within a range from 1 mm to 2 mm.

(2) In the partial leaflet resection and extension of the second embodiment of the present invention, the upper plate 2 of the template according to the present invention comprises a flat plate 6 having four sides. The length of each side of the flat plate 6 is defined by adopting, as a reference length, the length a of the side opposite the curved side of the flat plate 6. The length b, which is the distance between the center of the curved side and the center of the side opposite the curved side, is calculated by subtracting a value ranging from 1 mm to 5 mm from a value ranging from 0.65 to 0.70 times (subtracting 3 mm from about ⅔ times) the reference length a. The length c of each side adjacent to the curved side is calculated by subtracting a value ranging from 1 mm to 5 mm from a value ranging from 0.40 to 0.50 times (subtracting 3 mm from about 4/9 times) the length a of the side opposite the curved side.

Here, in order to provide a margin for suturing the artificial aortic valve leaflet when the artificial aortic valve leaflet is prepared, an extension part 8 is formed along the outer edges of the curved side of the flat plate 6 and the sides adjacent to the curved side. The extension part 8 is formed along the outer edges of the curved side of the flat plate 6 and the sides adjacent to the curved side so as to extend within a range from 1 mm to 2 mm.

(3) In the simple leaflet extension of the third embodiment of the present invention, the upper plate 2 of the template according to the present invention comprises a flat plate 6 having four sides. The length of each side of the flat plate 6 is defined by adopting, as a reference length, the length a of the side opposite the curved side of the flat plate 6. The length b, which is the distance between the center of the curved side and the center of the side opposite the curved side, is from 0.35 to 0.40 times (about 10/27 times) the reference length a. The length c of each side adjacent to the curved side is from 0.20 to 0.25 times (about 2/9 times) the length a of the side opposite the curved side.

Here, in order to provide a margin for suturing the artificial aortic valve leaflet when the artificial aortic valve leaflet is prepared, an extension part 8 is formed along the outer edges of the curved side of the flat plate 6 and the sides adjacent to the curved side. The extension part 8 is formed along the outer edges of the curved side of the flat plate 6 and the sides adjacent to the curved side so as to extend within a range from 1 mm to 2 mm.

The template of the present invention may include grips 10 if necessary. The grips 10 are coupled to both side of the upper and lower plates 2 and 4 of the template, and serve to fix the upper and lower plates 2 and 4 of the template so as to facilitate cutting a piece of pericardium when the artificial aortic valve leaflet is prepared. As long as the grips can accomplish this purpose, they are not limited to any shapes, and it is possible to use the same material as the upper and lower plates 2 and 4 of the template.

The uses of the artificial aortic valve leaflet and the template having the configuration described above will be described with reference to the surgical technique of the aortic valve leaflet, as follows.

(1) The use of the invention for the complete-leaflet replacement will be described below with reference to an example.

First, before the template of the present invention is used, the sizes of an aortic root and an individual aortic (valve) leaflet of a patient are determined. In the case of an adult, the shape of the normal aortic root is a truncated cone in which the ratio of an aortic annulus to the diameter of a sinotubular junction ranges from 1.0 to 1.2, and in which the ratio of the diameter of the distance from the center of the sinotubular junction to the center of the aortic annulus to the diameter of the sinotubular junction ranges from 0.8 to 1.0. Thus, in the case of a normal adult, it can be assumed that three aortic leaflets are uniformly disposed in the truncated cone.

A method of selecting the individual artificial aortic valve leaflet and the template for preparing the individual artificial aortic valve leaflet will be described with reference to an example. In the case in which the diameter of the sinotubular junction is set to 26 mm, the length a of the side opposite the curved side is set to 26 mm. The length b corresponding to the distance between the center of the curved side and the side opposite the curved side is set to 17.3 mm. The length of each of the two sides adjacent to the curved side is set to 11.5 mm. The extension part 8 is set to extend about 1.5 mm along the outer edges of the three sides except the side opposite the curved side.

The template of the present invention is preferably used along with a sinotubular junction repairing apparatus used for an “Aortic Valve Repairing Apparatus Set and Treatment Method

Using the Same” disclosed in Korean Patent No. 0466839, the application for which was filed, and which was granted to the applicant of the present invention. When the template having the same size as the sinotubular junction is used, the size of the template can be decided without individually measuring the sizes of the aortic valve leaflets of the patient.

When the size of the template is decided, a piece of biological material such as autologous pericardium, bovine or porcine pericardium or valve tissues, peritoneum, pleura or fascia of animal or man, which is fixed with glutaraldehyde in a flat shape, is disposed between the upper and lower plates 2 and 4, and then the upper and lower plates 2 and 4 are coupled with each other such that they are aligned with each other.

At this time, the coupled upper and lower plates 2 and 4 are fixed using the grips 10 so that they cannot move. Then, excess biological material extending beyond extension parts of the upper and lower plates 2 and 4 is cut using a pair of surgical scissors or a surgical knife. Thereby, the individual artificial aortic valve leaflet can be prepared.

Further, not all people have the same size aortic valve leaflets. In addition, some people inherently have only two aortic valve leaflets. Even in such cases, the size of each aortic valve leaflet is decided, and then the template having a proper size is used. Thereby, effective operation is possible.

The outer edges, expanded by the extension part 8, function to provide a margin for suturing when surgery is performed. When surgery is performed, the suture is made in a manner such that the ratio of the suturing interval of the aortic annulus of the patient to the suturing interval of the artificial aortic valve leaflet is set to 1:1.5 for a lower part ranging from the center of the curved side of the prepared artificial aortic valve leaflet to a one-third position, corresponding one third of the total length b, 1:1.2 for a straight line part ranging from the one-third point to the next one-third position, and 1:1 for a straight line part ranging from the next one-third position to the end. Thereby, the pericardium, which has a planar shape, can be converted into a container having a predetermined volume, like a sphere, and thus can perform the function of the aortic valve leaflet without any difficulty.

(2) The use of the invention for partial leaflet resection and extension will be described below with reference to an example. The aortic valve leaflet having a lesion is partially resected from the aortic annulus, with a margin ranging in size from about 1 mm to about 5 mm, preferably about 3 mm, left behind. Then, when the artificial aortic valve leaflet is prepared, the length c of each of the two sides adjacent to the curved side is reduced by an amount ranging from about 1 mm to about 5 mm, and preferably about 3 mm, and the length of the curved side and the length a of the side opposite the curved side have only to be set as in the complete-leaflet replacement. For example, when the diameter of the sinotubular junction is set to 26 mm, the reference length a of the side opposite the curved side is also set to 26 mm in the individual artificial aortic valve leaflet, and a size 26 template is selected. In this case, a modified template is selected as follows. More specifically, the modified template is configured so that the reference length a of the side opposite the curved side is set to 26 mm, so that the length b ranging from the center of the curved side to the side opposite the curved side is set to 14.3 mm, which is reduced by 3 mm compared to the same length of the template for complete-leaflet replacement, so that the length c of each side adjacent to the curved side is set to 8.5 mm, which is reduced by 3 mm compared to the same length of the template for complete-leaflet replacement, and so that the extension part 8 is increased by about 1.5 mm along the outer edges of the three sides except the side opposite the curved side.

When the size of the template is decided in this way, a piece of biological material such as autologous pericardium, the bovine or porcine pericardium or valve tissues, peritoneum, pleura or fascia of animal or man, which is fixed to glutaraldehyde in a flat shape, is disposed between the upper and lower plates 2 and 4, and then the upper and lower plates 2 and 4 are coupled with each other such that they are aligned with each other. At this time, the coupled upper and lower plates 2 and 4 are fixed using the grips 10 so that they cannot move. Then, excess biological material extending beyond extension parts of the upper and lower plates 2 and 4 is cut using a pair of surgical scissors or a surgical knife. Thereby, the individual artificial aortic valve leaflet can be prepared.

The outer edge, expanded by the extension part 8, functions to provide a margin for suturing when surgery is performed. Since about 3 mm of the remaining leaflet tissue is left behind, in contrast with the complete-leaflet replacement, the partial leaflet resection and extension enables easy suturing and relieves tension applied to a suturing thread at a sutured site.

(3) The use of the invention for the simple leaflet extension will be described below with reference to an example. In the case in which the leaflets are prolapsed, it is sufficient merely to reinforce the leaflet by extension, without resecting the leaflet. To this end, when the artificial aortic valve leaflet is prepared, the height (obtained by subtracting c from b) of the curved side alone has only to be reduced by about two thirds thereof, and the length c of each side adjacent the curved side has only to be reduced by about one half thereof.

For example, when the diameter of the sinotubular junction is set to 26 mm, the reference length a of the side opposite the curved side is also set to 26 mm in the individual artificial aortic valve leaflet, and a size 26 template is selected. In this case, a modified template is selected as follows. More specifically, the modified template is configured so that the reference length a of the side opposite the curved side is set to 26 mm, so that, among the length b from the center of the curved side to the side opposite the curved side, the height (=b−c) of the curved side alone is reduced by two thirds thereof and is set to 9.6 mm, so that the length c of each side adjacent to the curved side is reduced by one half thereof and is set to 5.7 mm, and so that the extension part 8 is increased by about 1.5 mm along the outer edges of the three sides, except the side opposite the curved side. When the size of the template is decided in this way, a piece of biological material such as autologous pericardium, bovine or porcine Pericardium or valve tissues, peritoneum, pleura or fascia of animal or man, which is fixed with glutaraldehyde in a flat shape, is disposed between the upper and lower plates 2 and 4, and then the upper and lower plates 2 and 4 are coupled with each other such that they are aligned with each other. At this time, the coupled upper and lower plates 2 and 4 are fixed using the grips 10 so that they cannot move. Then, excess biological material extending beyond the extension parts of the upper and lower plates 2 and 4 is cut using a pair of surgical scissors or a surgical knife. Thereby, the individual artificial aortic valve leaflet can be prepared. The outer edge, expanded by the extension part 8, functions to provide a margin for suturing when surgery is performed. Since the leaflet is not resected, the simple leaflet extension is very easily sutured.

As described above, it is to be appreciated that those skilled in the art can make substitutions, change or modify the embodiments into various forms without departing from the scope and spirit of the present invention. Accordingly, the foregoing embodiments should be regarded as illustrative rather than limiting. The scope of the present invention is not defined by the detailed description as set forth above but by the accompanying claims of the invention. It should also be understood that all alterations or modifications derived from the definitions and scopes of the claims and their equivalents fall within the scope of the invention.

### INDUSTRIAL APPLICABILITY

As described above, the present invention, not only is an artificial aortic valve leaflet for the aortic valve repair prepared in a simple and easy manner in a short time, artificial aortic valve leaflets having different sizes for patients who have various extents of damage or deformity to the aortic valve leaflet can also be prepared in a precise and easy manner, so that it can be used for the surgical treatment of reconstructing and repairing the dysfunctioning aortic valve leaflet in medical industrial fields.

1. An artificial aortic valve leaflet comprising a plane having four sides, three sides of which are linear and a remaining side of which is curved, wherein a distance between a center of the curved side and a center of the side opposite the curved side ranges from 0.35 to 0.70 times a length of the side opposite the curved side, and a length of each side adjacent to the curved side ranges from 0.20 to 0.50 times the length of the side opposite the curved side.

2. The artificial aortic valve leaflet according to claim 1, wherein the distance between the center of the curved side and the center of the side opposite the curved side ranges from 0.65 to 0.70 times the length of the side opposite the curved side, and the length of each side adjacent to the curved side ranges from 0.40 to 0.50 times the length of the side opposite the curved side.

3. The artificial aortic valve leaflet according to claim 1, wherein the length between the center of the curved side and the center of the side opposite the curved side is calculated by subtracting a value ranging from 1 mm to 5 mm from a value ranging from 0.65 to 0.70 times a length of the side opposite the curved side, and the length of each side adjacent to the curved side is calculated by subtracting a value ranging from 1 mm to 5 mm from a value ranging from 0.40 to 0.50 times the length of the side opposite the curved side.

4. The artificial aortic valve leaflet according to claim 1, wherein the distance between the center of the curved side and the center of the side opposite the curved side ranges from 0.35 to 0.40 times the length of the side opposite the curved side, and the length of each side adjacent to the curved side ranges from 0.20 to 0.25 times the length of the side opposite the curved side.

5. The artificial aortic valve leaflet according to claim 1, further comprising an extension part, which is formed along outer edges of the curved side and the sides adjacent to the curved side.

6. The artificial aortic valve leaflet according to claim 5, wherein the extension part has a width ranging from 1 mm to 2 mm

7. A template for use in the preparation of the artificial aortic valve leaflet comprising a plane having four sides, three sides of which are linear and the remaining side of which is curved, wherein the distance between a center of the curved side and a center of a side opposite the curved side ranges from 0.35 to 0.70 times a length of the side opposite the curved side, and a length of each side adjacent to the curved side ranges from 0.20 to 0.50 times the length of the side opposite the curved side.

8. The template for use in the preparation of the artificial aortic valve leaflet according to claim 7, wherein the distance between the center of the curved side and the center of the side opposite the curved side ranges from 0.65 to 0.70 times the length of the side opposite the curved side, and the length of each side adjacent to the curved side ranges from 0.40 to 0.50 times the length of the side opposite the curved side.

9. The template for use in the preparation of the artificial aortic valve leaflet according to claim 7, wherein the length between the center of the curved side and the center of the side opposite the curved side is calculated by subtracting a value ranging from 1 mm to 5 mm from a value ranging from 0.65 to 0.70 times the length of the side opposite the curved side, and the length of each side adjacent to the curved side is calculated by subtracting a value ranging from 1 mm to 5 mm from a value ranging from 0.40 to 0.50 times the length of the side opposite the curved side.

10. The template for use in the preparation of the artificial aortic valve leaflet according to claim 7, wherein the distance between the center of the curved side and the center of the side opposite the curved side ranges from 0.35 to 0.40 times the length of the side opposite the curved side, and the length of each side adjacent to the curved side ranges from 0.20 to 0.25 times the length of the side opposite the curved side.

11. The template for use in the preparation of the artificial aortic valve leaflet according to claim 7, further comprising an extension part, which is formed along outer edges of the curved side and the sides adjacent to the curved side.

12. The template for use in the preparation of the artificial aortic valve leaflet according to claim 11, wherein the extension part of the upper and lower plates has a width ranging from 1 mm to 2 mm.

13. The template for use in the preparation of the artificial aortic valve leaflet according to claim 11, wherein the grips are coupled to both side of the upper and lower plates and serve to fix the upper and lower plates.
